(19) United States
(12) Patent Application Publication
(10) Pub. No.: US 2008/0280311 A1
(43) Pub. Date: Nov. 13, 2008

(54) Immunoassay for the Simultaneous Immunochemical Determination of an Analyte (Antigen) and a Treatment Antibody Targeting the Analyte in Samples (Recovery Immunoassay)

(76) Inventor: Pavel Strohner, Berlin (DE)

Correspondence Address: BUCHANAN INGERSOLL & ROONEY PC, P.O. BOX 1404, ALEXANDRIA, VA, 22313-1404, US

(21) Appl. No.: 12/095,776

(22) PCT Filed: Nov. 29, 2006
(86) PCT No.: PCT/DE2006/002107

§ 371 Date: Jun. 24, 2008

(30) Foreign Application Priority Data
Dec. 2, 2005 (DE) 10 2005 057 920.5

(51) Int. Cl. G01N 33/00

(52) U.S. Cl. 435/7.94; 435/7.92

(57) ABSTRACT

The invention relates to an immunoassay for the simultaneous immunochemical determination of an analyte (antigen) and a treatment antibody targeting the analyte in samples (recovery immunoassay). The inventive immunoassay comprises an optionally marked catcher antibody bound to a surface, a marked treatment antibody, or a marked antibody which has the same binding epitope as the treatment body, an antigen that binds to the catcher antibody and the treatment antibody in the region of various epitopes, and in this way forms an immunochemical sandwich, a solution of the unmarked treatment antibody of a known concentration, and an antigen solution to be added to the samples. The invention can be applied to the fields of medical diagnosis, treatment control and pharmacological research.

The invention relates to an immunoassay for the simultaneous immunochemical determination of an analyte (antigen) and a treatment antibody targeting the analyte in samples (recovery immunoassay).

The invention can be applied to the fields of medical diagnosis, treatment control and pharmacological research.

### BACKGROUND

### A) Immunoassay

The immunoassay is a very common technique used to determine unknown concentrations of relevant analytical or diagnostic substances in sera, plasma, tissues, supernatants of tissues etc.

The immunoassays (radioimmunoassays (RIA), enzyme-immunoassays (EIA), luminescence-immunoassays (LIA) and similar assays) are based on antigen-antibody-reaction. A known reference antigen (standard) reacts with an antibody. There are assay variants in which antigens or antibodies are labelled. After running the assay the response of the bound labelled antigen or antibody (counts, optical density, relative light units) is measured. The relation between response and standard is described using a mathematical function or graphically using a standard curve.

An unknown sample is treated in the assay similar to the reference antigen. Its concentration can be determined from its response to the standard curve.

To avoid differences in the assay between standards and unknown samples, there are different techniques in use to prepare the unknown for the assay by extractions or substance addition or to prepare the standards by adding proteins, sera etc.

The correctness of these additions is checked by means of recovery experiments. Nowadays, software programs in measurement devices (Gamma-Counter, Reader) for immuno assay evaluation are very much standard equipment in medical ore biochemical laboratories.

Beyond that, there are immunoassay automates from different producers which combine automatic assay processing and data analysis.

All data analysis software for immunoassays are based on mathematical functions of standard curves, even if they are used as stored functions for several assay runs or as linear functions made from two standard concentrations

### B) Therapeutic Antibodies

Over the past few years a number of therapy antibodies have appeared on the market. Some of them are still being clinically tested. These antibodies are indicated for treating inflamatory and autoimmune diseases and cancer. These antibodies are originally monoclonal antibodies. The majority were gene-technically transferred to fully humanized antibodies or to chimeric antibodies (humane/mouse) to prevent immuno reactions against the drug. Table 1 shows a selection of these therapeutic antibodies. These antibodies are directed against disease-specific proteins to inhibit the disease affecting such proteins.

With a sandwich immunoassay you can measure the antigen, but its detectable concentration is influenced by the therapeutic antibody and special assay concepts (eg. Hamilton, R. G. et. al.) are needed to determine free and total antigen accurately.

TABLE 1

| Name of therapy | Researcher, | Type of | |
|---|---|---|---|
| antibody | Producer | antibody | Antigen; Indication |
| Omalizumab | Genentech; | humanized | IgE; Allergic Asthma |
| (Xolair) | Novartis; Tanox | | |
| Infliximab | Centocor | chimeric | TNF-α; rheumatoid |
| (Remicade) | | | arthritis, Crohn disease |
| Adalimumab | Cambridge | humanized | TNF-α; reumatoid |
| (Humira) | Antibody | | arthritis |
| | Technology; Abbott | | |
| Muromomab-CD3 | Ortho Biotech; | monoclonal | CD3; organ transplant |
| | Johnson&Johnson | | rejection |
| Daclizumab | Protein Design | humanized | CD25; kidney transplant |
| (Zenapax) | Labs | | rejection |
| Basiliximab | Novartis | chimeric | CD25; kidney transplant |
| (Simulect) | | | rejection |
| Raptiva | Genentech; | humanized | BLA^{a}, CD11a; Psoriasis |
| | Serono, Xoma | | |
| Natalizumab | Elan, Biogen | humanized | VLA-4β1^{b}; Crohn |
| (Antegren) | | | disease, multiple |
| | | | sclerosis |
| CDP-870 | Celltech; Pfizer | humanized | TNF-α; rheumatoid |
| | | | arthritis, Crohn disease |
| Trastuzumab | Genentech | humanized | HER-2/neu (p183^{neu}); |
| (Herceptin) | | | mammary cancer, lung |
| | | | cancer, pancreatic |
| | | | cancer |
| Daclizumab | Protein Design | chimeric | IL-2R; Leukemia |
| (Zenapax) | Labs | | |
| Endrecolomab | Johnson&Johnson | humanized | 17-A1; Colorectal |
| (Panorex) | | | cancer |

In the European patent EP 0850416B1 of 1 Aug. 2001, a method of two-dimensional determination of samples in immunoassay is described, which allows the analysis of the influence of cross reactive substances and matrix on immunoassay and the determination of correct antigen concentration.

Apart from that, there is no assay available which can correctly and simultaneously determine the antigen and the therapeutic antibody directed against the antigen.

The invention has the task of providing a simple assay which can simultaneously determine the concentration of antigen and therapeutic antibody directed against antigen in unknown samples, preferably for use in therapy control.

This task will be fulfilled according to claims with a special immunoassay and with a method for the application of this immunoassay.

The inventive immunoassay contains:
capture antibody, labelled (with biotin etc.) or unlabelled, immobilized at surface
labelled therapeutic antibody (with HRP, fluorescence dye, I-125 etc) or labelled antibody which binds to the same epitop of antigen as therapeutic antibody (signal antibody)
antigen of known concentrations which binds to capture and therapeutic antibody on different epitops and forms an immuno chemical sandwich.
unlabelled therapeutic antibody of known concentration
antigen of known concentrations for spiking unknown samples

### BASIC PRINCIPLE OF INVENTION

The starting point is a common sandwich-immunoassay consisting of capture antibody, antigen and signal antibody, as described above.

The presence of therapeutic antibody in samples causes a reduced recovery of antigen in the assay. The recovery of antigen in the assay and the concentration of therapeutic antibody have a strong correlation. This fact can be utilized to determine concentrations of therapeutic antibody and free and total antigen in unknown samples.

For this reason this immunoassay can be called recovery-immunoassay.

### Procedure:

You have to develop a common sandwich-immunoassay with
a) a capture antibody which is immobilized at biochip surface or micro plate etc.
b) an antigen, a protein or another biopolymer with more than one binding epitop measured as standard or sample in immunoassay
c) a signal antibody (therapeutic antibody or antibody which binds to the same epitop), labelled radioactive or nonradioactive. The nonradioactive labelling can be done using common procedures with horseradish peroxidase, alcalic phosphatase, luminiscence and fluorescence labels.

The above described sandwich-immunoassay then proceeds with or without the addition of 2 to 5 different concentrations of therapeutic antibody.

For the assay without therapeutic antibodies it is important that it correctly determines samples without therapeutic antibody.

The response values of the 2 up to 6 immunoassays is analysed regarding the reduction of response, and consequently the reduction of recovered antigen concentration dependent on the added therapeutic antibody.

The connection between the recovery of response resp. recovery of recalculated antigen concentration and added therapeutic antibody is reduced by regression to a appropriate mathematical function (recovery function).

In the case of excess of capture antibody and labelled antibody there is a linear correlation between reciprocal of recovery and added therapeutic antibody.

The precondition of the validity of recovery function is that the immunoassay is not distorted by matrix effects. You have to guarantee the similarity of reaction media in standards and samples.

Unknown samples are spiked with 2 to 3 different known concentrations of antigen (standards). In the assay you have to analyse the recovery of known antigen in an unknown sample. Should the recovery be around 100%, then no therapeutic antibody is in the sample. If the recovery is lower than 100% you can estimate the concentration of therapeutic antibody with the recovery function described above.

Currently there are no—or at least only time- and material consuming—methods available for the therapy control of therapeutic antibodies.

For instance, Hamilton R. G. et al. needs two different very special assays to determine free and total IgE in sera of Omalizumab-treated patients, but without determining Omizumab itself.

With the use of the inventive recovery immunoassay you can very easily run therapy control for Omalizumab treatment in sera of patients with asthma.

The following two examples will explain the recovery immunoassay (here ELISA=Enzyme-linked immuno sorbant assay) in greater detail.

### EXAMPLE 1

The effect of adding therapeutic antibody Omalizumab (Xolair=E-25) on the IgE-ELISA in buffer on Streptavidine-coated microtiter plates

### Material:

Streptavidine-coated microtiter plate, 96 cavities, SC-coating (company BioTeZ)
Capture biotin-labelled antibody, concentrate B 216/290702; anti-human IgE, mAb E-411
IgE-standard, IgE from OEM
HRP-labelled Omalizumab (PD08)
Therapeutic antibody Omalizumab (Xolair)=E-25; Novartis
Coating buffer (PBS 0.05M, pH=7.2; 0.1% BSA)
Reaction buffer ((PBS/0.33% Casein+0.0125% TWEEN 20)
Washing dilution (0.9% NaCl/0.1% Tween)
Chromogen-dilution Orthophenylendiamin (OPD)

Table 2 shows the assay design on microtiter plate and in table 3 the results of the assay.

### Course of Assay

1. Immobilization of capture antibody: Addition of 200 μl biotin-labelled antibody, B 216/290702 (4 μg/ml) per well of microtiter plate (MTP). Incubation over night at 4° C.
2. The following day, the MTP wells are washed twice using washing dilution in an MTP-washer.
3. Preparation IgE standard dilutions 0/3.125/6.25/12.5/25/50/100/200 ng IgE/ml according to the assay design.
4. Preparation of E-25 dilutions 0/0.0625/0.125/0.25/0.5/1 μg/ml according to assay design
5. Mixing of 50 μl of dilutions 3. and 4. to a initial solution
6. Mixing of initial solution (100 μl) with HRP-conjugate (PD08, 100 ng/ml, 100 μl) to a reaction solution
7. Addition of 200 ml reaction solution of MTP per well according to assay design
8. Incubation for 3 hours at room temperature
9. Washing MTP wells twice using washing dilution in MTP-washer
10. Enzyme reaction with OPD (14 mg OPD in 20 ml buffer+80 μl 3% H₂O₂), Adding 200 μl OPD-solution and stopping enzyme reaction after 30 minutes with 50 μl H₂SO₄ [5M]

TABLE 2
Assay design on Streptavidine-coated microtiter plate:

| | | All estimations as duplicates | | | | | | | | | | | |
|---|---|---|---|---|---|---|---|---|---|---|---|---|---|
| | | 1 | 2 | 3 | 4 | 5 | 6 | 7 | 8 | 9 | 10 | 11 | 12 |
| Zeile | | E-25-conc. (μg/ml) | | | | | | | | | | | |
| Spalte | IgE-conc. ng/ml | 0 | 0 | 0.033 | 0.033 | 0.063 | 0.063 | 0.125 | 0.125 | 0.25 | 0.25 | 0.5 | 0.5 |
| A | 0 | | | | | | | | | | | | |
| B | 1.5625 | | | | | | | | | | | | |
| C | 3.125 | | | | | | | | | | | | |
| D | 6.25 | | | | | | | | | | | | |
| E | 12.5 | | | | | | | | | | | | |
| F | 25 | | | | | | | | | | | | |
| G | 50 | | | | | | | | | | | | |
| H | 100 | | | | | | | | | | | | |

TABLE 3
Result of assay
Mean values of duplicates as Optical Density (O.D.)

| | | E-25-conc. in μg/ml | | | | | |
|---|---|---|---|---|---|---|---|
| | | 0 | 31.250 | 62.5 | 125 | 250 | 500 |
| | IgE-conc. ng/ml | O.D. | O.D. | O.D. | O.D. | O.D. | O.D. |
| A | 0 | 0.070 | 0.064 | 0.054 | 0.054 | 0.052 | 0.064 |
| B | 1.563 | 0.154 | 0.149 | 0.143 | 0.112 | 0.102 | 0.104 |
| C | 3.125 | 0.201 | 0.192 | 0.169 | 0.145 | 0.127 | 0.119 |
| D | 6.25 | 0.277 | 0.266 | 0.256 | 0.186 | 0.156 | 0.146 |
| E | 12.5 | 0.493 | 0.501 | 0.434 | 0.365 | 0.290 | 0.244 |
| F | 25 | 0.984 | 0.879 | 0.805 | 0.707 | 0.585 | 0.382 |
| G | 50 | 1.482 | 1.257 | 1.198 | 0.948 | 0.751 | 0.587 |
| H | 100 | 2.378 | 2.285 | 2.216 | 1.927 | 1.432 | 1.134 |

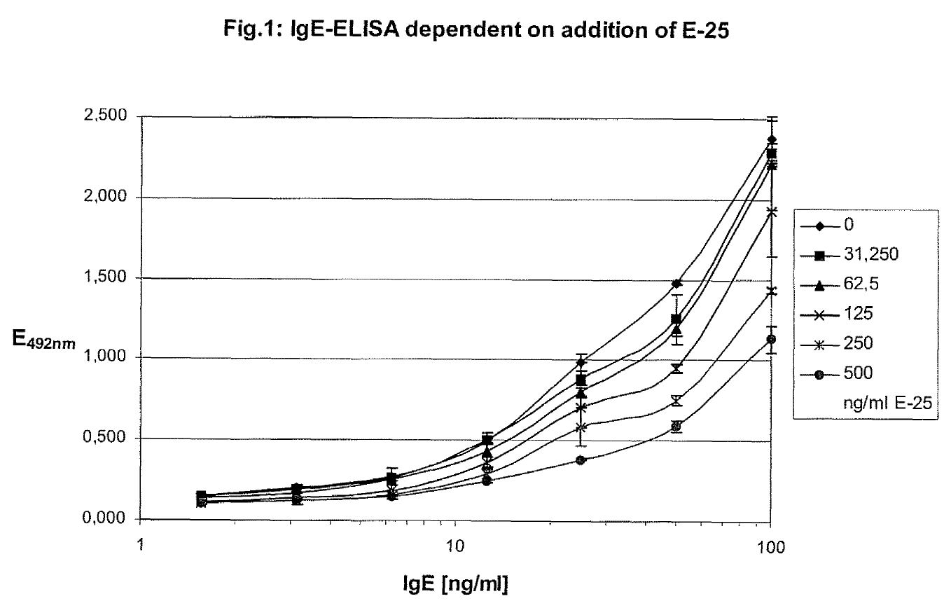

FIG. 1. illustrated the IgE-ELISA dependent on E-25-addition.

If you look at the response values in relation to the addition of therapeutic antibody E-25, you find a simple linear correlation (Table 4)

TABLE 4

| | Addition of E-25 to IgE standard solutions in μg/ml | | | | | |
|---|---|---|---|---|---|---|
| | 0 | 0.03125 | 0.0625 | 0.125 | 0.25 | 0.5 |
| Coefficient of determination for the | 1.000 | 0.994 | 0.993 | 0.984 | 0.990 | 0.981 |
| linear correlation with the IgE | | | | | | |
| standards without E-25 | | | | | | |
| Recovery of O.D. of the IgE | 100.00% | 93.05% | 90.32% | 77.62% | 57.77% | 44.06% |
| standards | | | | | | |

Clearly all response values of IgE standard curves with the addition of E-25 are linearly correlated to the response values of IgE standard curve without E-25.

The recovery of O.D. is systemtically reduced dependent on the addition of E-25.

This fact can be reduced to a simple linear function

1/recovery=1+2.626*Y (equation 1)

Y: concentration of therapeutic antibody E-25

The coefficient of determination for the linear correlation between the inverse of recovery value and the concentration of therapeutic antibody E-25 was 0.9907.

The immunoassay is made to determine unknown antigen concentrations.

The starting point for this purpose is the solution of known antigen concentration as so-called standard curves. For these standard curves you run the assay to obtain response values (eg. O.D.). The connection between known antigen concentration and response values is usually reduced via regression to a mathematical function as

Logit(E)=ln((E−NSB)/(Eₘₐₓ−E+NSB))=a+b*ln(X) (equation 2)

ln: natural logarithmus
X: antigen concentration

### E: O.D.

NSB: nonspecific binding

### Eₘₐₓ: Maximal O.D.

a; b: parameter of funktion

For our example we obtained by linear regression for the IgE standard curve without adding therapeutic antibody (E-25):

Logit(E)=0.892*ln(IgE)−5.30775 (equation 3)

The coefficient of determination for the linear correlation was 99.292%, which means that the function conforms with the experimental values of 99.3%.

To determine the antigen concentration of the unknown samples the inverse function of equation 3 is needed:

ln(IgE)=(5.30775+Logit(E))/0.892 (equation 4)

With equation 4 we then determined from the response values (O.D.) in the standard curves with the addition of E-25 the antigen concentration (IgE concentration, table 5).

TABLE 5

| | Addition of E-25 μg/mL | | | | | |
|---|---|---|---|---|---|---|
| | 0 | 0.03125 | 0.0625 | 0.125 | 0.25 | 0.5 |
| | IgE, | IgE, | IgE, | IgE, | IgE, | IgE, |
| IgE ng/ml | recalc. | recalc | recalc | recalc | recalc | recalc |
| 1.56 | 1.81 | 1.69 | 1.56 | 0.82 | 0.62 | 0.66 |
| 3.13 | 3.02 | 2.77 | 2.20 | 1.61 | 1.17 | 0.99 |
| 6.25 | 5.09 | 4.78 | 4.49 | 2.63 | 1.87 | 1.62 |
| 12.50 | 11.61 | 11.87 | 9.74 | 7.63 | 5.46 | 4.17 |
| 25.00 | 29.25 | 25.19 | 22.41 | 18.85 | 14.64 | 8.14 |
| 50.00 | 50.74 | 40.55 | 38.03 | 27.82 | 20.43 | 14.72 |
| 100.00 | 99.60 | 93.84 | 89.64 | 73.20 | 48.41 | 35.32 |
| Coefficient of | | 0.994 | 0.994 | 0.985 | 0.988 | 0.993 |
| determination | | | | | | |
| Recovery | | 92.23% | 88.50% | 71.91% | 47.86% | 34.74% |

IgE-recalc.: IgE values determined by equation 4 from O.D. in table 3

As expected, we also found, as in the case of response values (O.D.), a systematic decrease of recovery dependent on the addition of the therapeutic antibody E-25 in the recalculated IgE concentrations. The IgE concentration in standard curves with the addition of E-25 were also linearly correlated to the IgE concentration in the standard curve without E-25 (in all cases more than 98.5%).

Between recovery values and concentration of therapeutic antibody E-25 we found via linear regression, as in the O.D. values, the simple linear function

1/recovery=0.9606+3.932*Y (equation 5)

Y: concentration of therapeutic antibody E-25

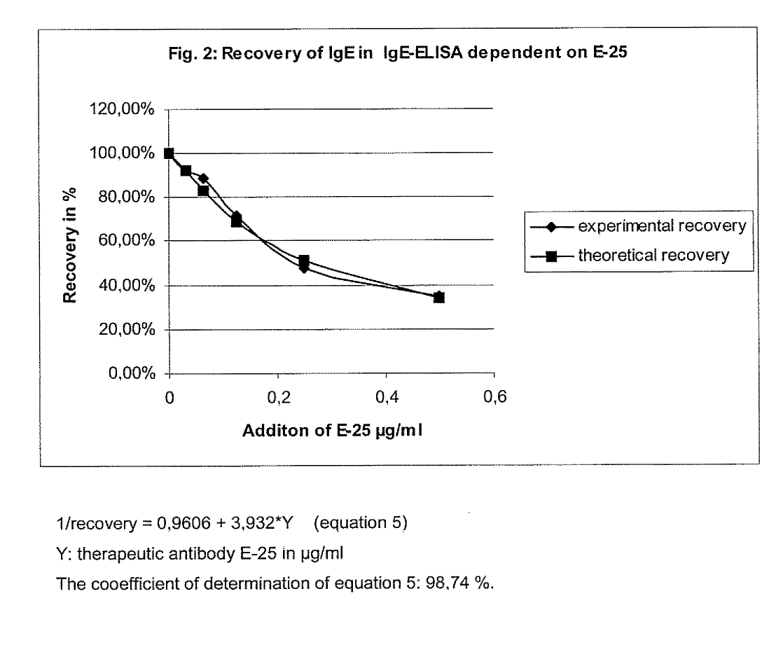

The coefficient of determination for this function was 98.74%. FIG. 2 shows good conformity between experimental and theoretical values.

With the inverse of this function it is possible to determine the content of therapeutic antibody in unknown samples.

Example 1 was needed to show the basic principle of recovery ELISA using the example of IgE-ELISA influenced by therapeutic antibody E-25 (Xolair=Omalizumab) in buffer solution.

In example 2 we will show how to determine free and total antigen (IgE) and therapeutic antibody (E-25) with the principles of example 1 and the simple spiking of unknown samples with antigen (IgE).

To guarantee the conformity of reaction solutions of standards and unknown sera we prepared IgE free sera via immuno affinity chromatography and have used these IgE free sera as solutions for the standards.

### EXAMPLE 2

The effect of adding therapeutic antibody Omalizumab (Xolair=E-25) on the recovery of unknown sera in IgE ELISA on streptavidine-coated microtiter plates and determination of therapeutic antibody E-25 in unknown sera, spiked with E-25

### Material:

Streptavidine-coated microtiter plate, 96 cavities, SC-coating (company BioTeZ)
Capture biotin-labelled antibody, concentrate B 216/290702; anti-human IgE, mAb E-411
IgE-standard, IgE from OEM
HRP-labelled Omalizumab (PD08)
Therapeutic antibody Omalizumab (Xolair)=E-25; Novartis
Coating buffer (PBS 0.05M, pH=7.2; 0.1% BSA)
Reaction solution of standard curves (IgE free serum diluted 1:10 in PBS/0.33% Casein+0.0125% TWEEN 20)
Unknown sera (diluted 1:10 in reaction buffer: PBS/0.33% Casein+0.0125% TWEEN 20)
Washing dilution (0.9% NaCl/0.1% Tween)
Chromogen-dilution Orthophenylendiamin (OPD)

Table 6 and 7 shows the assay design on the microtiter plate and table 8-10 the results of the assay.

TABLE 6
Assay design for the standard curves (first half of microtiter plate)

| | | Standard curves | | | | | |
|---|---|---|---|---|---|---|---|
| | | All determinations as duplicates | | | | | |
| | | 1 | 2 | 3 | 4 | 5 | 6 |
| Row | | E-25 (μg/ml) | | | | | |
| Column | IgE-Konz. μg/ml | 0 | 0 | 0.125 | 0.125 | 0.5 | 0.5 |
| A | 0 | | | | | | |
| B | 1.5625 | | | | | | |
| C | 3.125 | | | | | | |
| D | 6.25 | | | | | | |
| E | 12.5 | | | | | | |
| F | 25 | | | | | | |
| G | 50 | | | | | | |
| H | 100 | | | | | | |

TABLE 7
Assay design for the unknown sera
(second half of microtiter plate)

| | | Unknown sera | | | | | |
|---|---|---|---|---|---|---|---|
| | | All determinations as duplicates | | | | | |
| | | 7 | 8 | 9 | 10 | 11 | 12 |
| Row | | Spiking of unknown serum with IgE in ng/ml | | | | | |
| Column | Unknown serum | 0 | 0 | 6.25 | 6.25 | 25 | 25 |
| A | Serum 3 | | | | | | |
| B | Serum 5 | | | | | | |
| C | Serum 6 | | | | | | |
| D | Serum 7 | | | | | | |
| E | Serum 3 + | | | | | | |
| | 0.031 μg/ml E-25 | | | | | | |
| F | Serum 5 + | | | | | | |
| | 0.063 μg/ml E-25 | | | | | | |
| G | Serum 6 + | | | | | | |
| | 0.125 μg/ml E-25 | | | | | | |
| H | Serum 7 + | | | | | | |
| | 0.25 μg/ml E-25 | | | | | | |

### Course of Assay

1. Immobilization of capture antibody: Addition of 200 μl biotin-labelled antibody, B 216/290702 (4 μg/ml) per well of microtiter plate (MTP). Incubation over night at 4° C.
2. The following day the MTP wells are washed twice using washing dilution in MTP-washer.

### 3. Standards

For Rows A-H, Columns 1-6: 0/3.125/6.25/12.5/25/50/100/200 ng IgE/ml and 0/0.125/0.5 μg/ml E-25 in reaction solution of standard curves (1:10-diluted IgE-free sera)

### 4. Unknown Sera

| | | |
|---|---|---|
| Column 7-12, A-D | Serum 3, 5, 6, 7, 1:10- | Spiked with 0/6.25/25 |
| | diluted in Buffer | ng/ml IgE |
| Spalte 7-12, E | Serum 3 1:10-diluted in | Spiked with 0/6.25/25 |
| | Buffer + 0.031 μg/ml E-25 | ng/ml IgE |
| Spalte 7-12, F | Serum 5 1:10-diluted in | Spiked with 0/6.25/25 |
| | Buffer + 0.063 μg/ml E-25 | ng/ml IgE. |
| Spalte 7-12, G | Serum 6 1:10-diluted in | Spiked with 0/6.25/25 |
| | Buffer + 0.125 μg/ml E-25 | ng/ml IgE. |
| Spalte 7-12, H | Serum 7 1:10-diluted in | Spiked with 0/6.25/25 |
| | Buffer + 0.25 μg/ml E-25 | ng/ml IgE |

5. Incubation for 3 hours at room temperature
6. Washing MTP wells twice with washing dilution in MTP-washer
7. Enzyme reaction with OPD (14 mg OPD in 20 ml buffer+80 μl 3% H₂O₂), Addition of 200 μl OPD-solution and stopping enzyme reaction after 30 minutes with 50 μl H₂SO₄ [5M]

### Table 8: Result of Immunoassay:

a) Standard curves (column 1-6)

TABLE 8
Result of Immunoassay:
a) Standard curves (column 1-6)

| | Addition of E-25 in μg/ml | | |
|---|---|---|---|
| | 0 | 0.125 | 0.5 |
| ng/ml IgE | O.D. | O.D. | O.D. |
| 0 | 0.045 | 0.039 | 0.050 |
| 1.5625 | 0.098 | 0.096 | 0.074 |
| 3.125 | 0.121 | 0.102 | 0.084 |
| 6.25 | 0.170 | 0.150 | 0.110 |
| 12.5 | 0.281 | 0.264 | 0.195 |
| 25 | 0.546 | 0.439 | 0.282 |
| 50 | 0.921 | 0.827 | 0.421 |
| 100 | 1.602 | 1.316 | 0.798 |
| Coefficient of | | 0.9968 | 0.9856 |
| determination | | | |
| Recovery | 1 | 0.8283 | 0.4671 |

O.D.: Optical Density

As expected, we found a systematic decrease in O.D in the IgE standard curves dependent on the addition of E-25.

The correlation of antigen concentration (IgE) and the response values O.D. was reduced by linear regression using common logit-log-function (equation 3) for determination of unknown sera. By using inverse of function 3.

By regression we found

Logit(E)=0.8984*ln(IgE)−5.9079. (equation 6)

The inverse of this function

ln(IgE)=(5.39079+Logit(E))/0.8984) (equation 7)

was used to recalculate the standard curves by adding E-25 and the unknown sera. For the standard curves we obtained by recalculation IgE concentrations shown in table 9:

TABLE 9

| | Addition of E-25 μg/ml | | |
|---|---|---|---|
| | 0 | 0.0625 | 0.25 |
| | Recalculated IgE | Recalculated IgE | Recalculated IgE |
| IgE concentration in ng/ml | conc. in ng/ml | conc. in ng/ml | conc. in ng/ml |
| 0 | | | |
| 1.5625 | 1.91 | 1.82 | 0.88 |
| 3.125 | 2.95 | 2.08 | 1.30 |
| 6.25 | 5.29 | 4.29 | 2.42 |
| 12.5 | 11.02 | 10.11 | 6.54 |
| 25 | 26.48 | 20.02 | 11.10 |
| 50 | 51.62 | 44.98 | 18.93 |
| 100 | 101.94 | 81.97 | 43.01 |
| Recovery of IgE standard | 102.709% | 83.349% | 42.050% |
| Coefficient of determination | 99.938% | 99.721% | 99.464% |

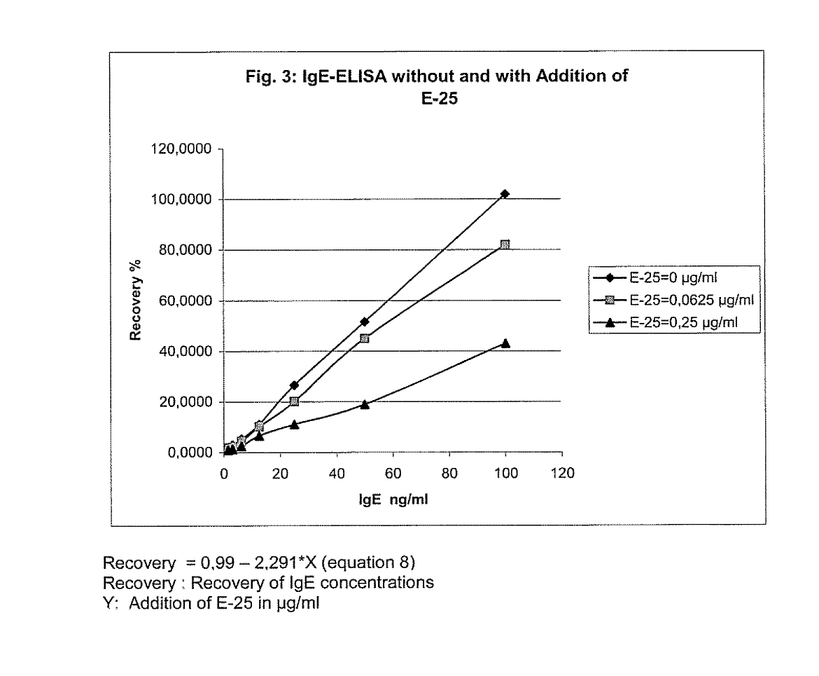

We found a systematic decrease in IgE concentrations dependent on the addition of E-25 (see FIG. 3), which we were able to reduce in a simple linear function for the dependence of recovery on the concentration of therapeutic antibody E-25.

recovery=0.99−2.291*Y (equation 8).

Y: Addition of E-25 in μg/ml

The coefficient of determination for this linear function was 99.84%.

For the unknown sera we obtained in ELISA results as shown in table 10.

TABLE 10
Anonymous sera without and with addition of E-25

| | Spiking with IgE(ng/ml) [symbol] | | | | | | | |
|---|---|---|---|---|---|---|---|---|
| | | | | | Unspiked | | Addition of | |
| | | | | | IgE value | Coefficient of | E-25 in μg/ml | Recalculated |
| | 0.00 | 6.25 | 25.00 | recovery | ng/ml | determination | [symbol] | E-25 μg/ml |
| Serum 3 | | | | | | | | |
| Recalculated IgG in | 10.54 | 20.97 | 38.60 | 108.04% | 12.114 | 0.982 | 0.000 | |
| ng/ml | | | | | | | | |
| Expected value | 12.11 | 16.79 | 35.54 | | | | | |
| Recalculated IgG in | 7.46 | 12.86 | 32.84 | 102.68% | 7.021 | 0.998 | 0.031 | 0.01 |
| ng/ml | | | | | | | | |
| Expected value | 7.02 | 13.71 | 32.46 | | | | | |
| Serum 5 | | | | | | | | |
| Recalculated IgG in | 14.70 | 23.92 | 41.06 | 102.19% | 15.915 | 0.988 | 0.000 | |
| ng/ml | | | | | | | | |
| Expected value | 15.92 | 20.95 | 39.70 | | | | | |
| Recalculated IgG in | 12.28 | 15.86 | 29.91 | 71.54% | 11.897 | 0.998 | 0.063 | 0.08 |
| ng/ml | | | | | | | | |
| Expected value | 11.9 | 18.53 | 37.28 | | | | | |
| Serum 6 | | | | | | | | |
| Recalculated IgG in | 4.08 | 9.63 | 27.98 | 96.14% | 3.880 | 1.000 | 0.000 | |
| ng/ml | | | | | | | | |
| Expected value | 3.88 | 10.33 | 29.08 | | | | | |
| Recalculated IgG in | 3.39 | 8.00 | 16.52 | 50.89% | 4.002 | 0.988 | 0.125 | 0.18 |
| ng/ml | | | | | | | | |
| Expected value | 4.00 | 10.64 | 29.39 | | | | | |
| Serum 7 | | | | | | | | |
| Recalculated IgG in | 3.67 | 7.36 | 26.82 | 95.20% | 2.700 | 0.991 | 0.000 | |
| ng/ml | | | | | | | | |
| Expected value | 2.70 | 9.92 | 28.67 | | | | | |
| Recalculated IgG in | 2.97 | 5.36 | 12.94 | 40.00% | 2.924 | 1.000 | 0.250 | 0.27 |
| ng/ml | | | | | | | | |
| Expected value | 2.94 | 9.22 | 27.97 | | | | | |

### Explanation:

In assay, we used 4 sera of unknown healthy blood donors. All sera were spiked with 6.25 and 25 ng/ml IgE standard. We also spiked the sera with different therapeutic antibody E-25 to simulate sera of E-25-treated patients.

To calculate the antigen concentrations (IgE) from the O.D. we used the inverse of the function of the standard curve without E-25: ln(IgE)=(5.39079+Logit(E))/0.8984) (equation 7).

We obtained the following detailed results:

### 1. Serum 3:

Serum 3 was spiked with 6.25 and 25 ng/ml IgE, Without E-25 and a initial value of 12 ng/ml IgE for the serum unspiked with IgE we obtained a recovery of 108%. This recovery was not significantly different from the expected recovery of 100%.

The addition of 0.031 μg/ml E-25 (Xolair) showed no significant decrease in recovery.

### 2. Serum 5:

Serum 5 was spiked with 6.25 and 25 ng/ml IgE, Without E-25 and a initial value of 15.9 ng/ml IgE for the serum unspiked with IgE we obtained a recovery of 102%. This recovery was not significantly different from the expected recovery of 100%.

The addition of 0.063 μg/ml E-25 (Xolair) showed a significant decrease in recovery to 72%. With equation 8 we calculated a E-25 concentration of 0.08, which was near to the expected E-25 concentration of 0.063 μg/ml.

### 3. Serum 6:

Serum 6 was spiked with 6.25 and 25 ng/ml IgE, Without E-25 and a initial value of 3.9 ng/ml IgE for the serum unspiked with IgE we obtained a recovery of 96%. This recovery was not significantly different from the expected recovery of 100%.

The addition of 0.125 μg/ml E-25 (Xolair) showed significant decrease in recovery to 50%. With equation 8 we calculated a E-25 concentration of 0.18, which was near to the expected E-25 concentration of 0.125 μg/ml.

### 4. Serum 7:

Serum 6 was spiked with 6.25 and 25 ng/ml IgE, without E-25 and a initial value of 2.7 ng/ml IgE for the serum unspiked with IgE we obtained a recovery of 95%. This recovery was not significantly different from the expected recovery of 100%.

The addition of 0.25 μg/ml E-25 (Xolair) showed a significant decrease in recovery to 40%. With equation 8 we calculated a E-25 concentration of 0.27, which was near to the expected E-25 concentration of 0.25 μg/ml.

### REFERENCES

Rodbard D., Ratford P. L., Cooper J. and Ross G. T. (1968). Statistical quality control of radioimmunoassays. J. Clin. Endocrinol. Metab. 29, 352.
Azimzadeh A., Pellequer J. L. and Van Regenmortel M. H. V. (1992). Operational aspects of antibody affinity constants measured by liquid-phase and solid-phase assays. J. Molecular Recognition. 5, 9.
Goldberg M. E. and Djavadi-Ohaniance L. (1993). Methods for measurement of antibody/antigen affinity based on ELISA and RIA. Current Opinion in Immunology 5, 278.
Hamilton R. G., Marcotte G. V., Saini S. S.: Immunological methods for quantifying free and total serum Ige levels in allergy patients receiving omalizumab (Xolair) therapy. J. Immunol. Methods. 2005, 303 (1-2):81-91
Europäischen Patentschrift EP 0850416B1 from 01.08.2001

1. Immunoassay for the simultaneous immunochemical determination of an analyte (antigen) and a treatment (therapeutic) antibody targeting the analyte in samples (recovery immunoassay) containing
capture antibody, labelled (with biotin etc.) or unlabelled, immobilized at surface
labelled therapeutic antibody (with HRP, fluorescence dye, I-125 etc) or labelled antibody which binds to the same epitop of antigen as therapeutic antibody (signal antibody)
antigen of known concentrations, which binds to capture and therapeutic antibody on different epitops and forms an immuno chemical sandwich.
unlabelled therapeutic antibody of known concentration
antigen of known concentrations for spiking unknown samples

2. Method for the simultaneous immunochemical determination of an analyte (antigen) and a treatment (therapeutic) antibody targeting the analyte in samples with an immunoassay according to claim 1, used in such a way that
Sandwich-Immunoassay standard curves are made without and also with the addition of 1 up to 4 different concentrations of therapeutic antibody.
The reduction of recovered antigen concentration dependent on the addition of therapeutic antibody is analysed by statistical regression analysis.
Samples with unknown antigen concentration are spiked with 2 up to 3 known antigen concentrations. The recovery of the spiked samples are determined.
From the therapeutic antibody concentration derived from the recovery values in the standard curves the therapeutic antibody concentration in the samples is determined, derived from its recovery values. The free antigen concentrations are determined in the unspiked samples. The total antigen concentration from the free antigen concentration can be derived with the recovery value.
